(19) United States
(12) Patent Application Publication
(10) Pub. No.: US 2024/0261152 A1
(43) Pub. Date: Aug. 8, 2024

(54) EAR PLUG

(71) Applicant: Milton Stuart Jones, Golden Valley, AZ (US)

(72) Inventor: Milton Stuart Jones, Golden Valley, AZ (US)

(21) Appl. No.: 18/105,173

(22) Filed: Feb. 2, 2023

(51) Int. Cl. A61F 11/08

(52) U.S. Cl. CPC A61F 11/085

(57) ABSTRACT

An ear plug that is adapted to regulate air pressure and protect the inner ear from various weather conditions. The ear plug is formed from of a combination of breathable sterile medical grade cotton gauze and a sterile fiber tissue filler, and which is formed and adapted for a comfortable fit adjacent to or within the ear canal of a user and can also provide noise reduction capabilities.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever 37 CFR 1.71(d).

### BACKGROUND OF THE INVENTION

### 1. Field of the Invention

This invention relates generally to ear plugs, and more specifically to ear plugs adapted to provide protection from weather conditions and including noise reduction capabilities.

### 2. Description of the Related Art

Prior art ear plugs are usually formed from one material and adapted to seal an ear opening and are generally for noise reduction. These prior art ear plugs are usually very uncomfortable to wear for long periods of time and create soreness and inner ear heating discomfort. Furthermore, these prior art ear plugs do not help regulate air pressure nor protect the inner ear from various weather conditions. Accordingly, the present invention overcomes the disadvantages associated with the prior art.

### BRIEF SUMMARY OF THE INVENTION

In view of the foregoing disadvantages inherent in the known types of ear plugs or the like in the prior art, the present invention provides an ear plug is formed from of a combination of breathable sterile medical grade cotton gauze and a sterile fiber tissue filler, and which is formed and adapted for a comfortable fit and can provide noise reduction capabilities.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

### BRIEF DESCRIPTION OF THE DRAWINGS

The figures which accompany the written portion of this specification illustrate embodiments according to the teachings of the present invention.

FIG. 1 shows a side view of the instant ear plug according to the preferred embodiment of the present invention.

FIG. 2 shows a bottom view of the instant ear plug according to the preferred embodiment of the present invention of FIG. 1.

FIG. 3 shows the instant ear plug being placed within the ear canal of a user's ear according to the preferred embodiment of the present invention of FIG. 1.

The various embodiments of the present invention will hereinafter be described in conjunction with the appended drawings.

### DETAILED DESCRIPTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

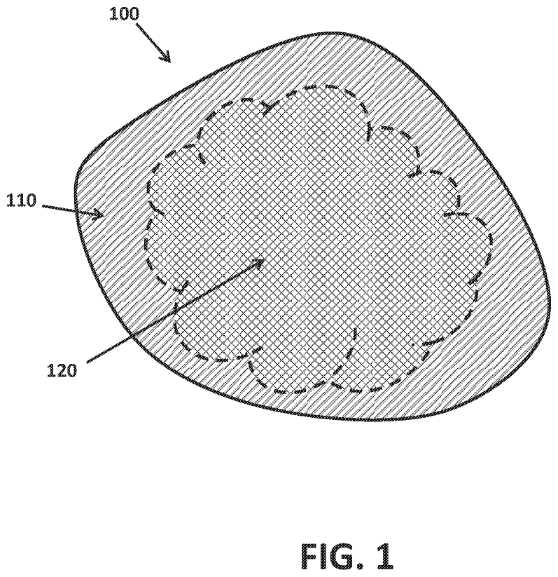

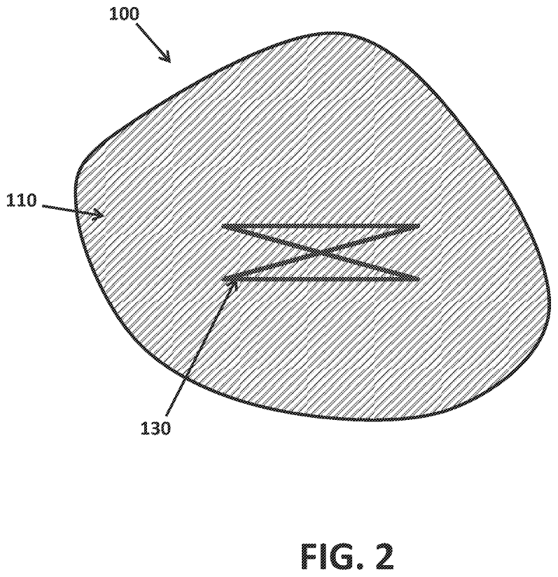

Turning now descriptively to drawing, referring to FIGS. 1-3, the present invention discloses an improved ear plug 100 comprising an outer layer 110 formed from a gauze material, and an inner filler material 120 formed from a fiber material, wherein the inner filler material is surrounded by the outer layer and enclosed therein, wherein the improved ear plug is formed having a predetermined size, shape, and stiffness, and wherein the improved ear plug is adapted to be removably placed adjacent to or within the ear canal of a user's ear and adapted to prevent and relieve symptoms caused by changes in barometric air pressure, cold temperatures, or windy conditions.

In the preferred embodiment, the gauze material may be formed as a breathable sterile medical grade cotton gauze material, and a sterile fiber tissue filler material. The tissue fiber material can be formed from wadded up toilet paper. The gauze material and the inner filler material are sized and shaped such that the inner filler material can be wrapped within the gauze material, and then edges of the gauze material connected together forming a knot 130 thereby resulting in said predetermined size, shape, and stiffness.

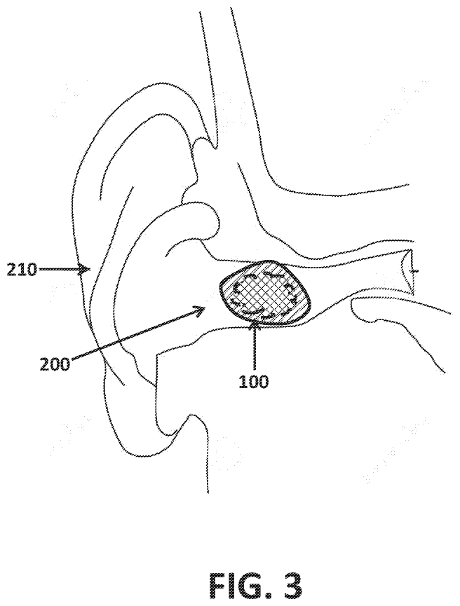

In use, as shown in FIG. 3, the instant ear plug 100 is best used when barometric pressure changes, for example when a person's position changes in altitude. The lowering of barometric pressure is usually the change that triggers symptoms. In those situations a person may experience discomforts, such as ear pain, headaches, facial pain, sinus pain, and eye pain.

The instant ear plug is also best used during cold or windy conditions, including indoor air conditioning and window drafts. To relieve these discomforts the instant ear plug 100 is inserted adjacent to or within the user's ear canal 210 of their ear 200, allowed to expand therein blocking outside pressure, temperature changes, and wind, and kept therein until the symptoms dissipate. The instant ear plug also provides a degree of noise reduction, such that a user can use the instant ear plug for that purpose as well.

The method of making the instant ear plug 100 can be done in many ways. By hand, a user can wrap the inner filler material 120 within the gauze material 110, and then twist the edges of the gauze creating a knot 130 resulting in an ear plug having the desired size, shape, and stiffness. In mass production, the instant ear plug can be created using machines to create an interior opening within individual gauze pads, then stuffing inner filler material therein, then closing and sealing the gauze pads to predetermined sizes, shapes, and stiffness. Or, the instant ear plug can be created using machines to wrap inner filler material within gauze material, and then twisting the edges of the gauze material creating a knot resulting in an ear plug having the desired size, shape, and stiffness. Other methods and processes can also be used and are inherently disclosed herein.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

What is claimed is:

1. An improved ear plug comprising:
an outer layer;
wherein said outer layer is formed from a gauze material; and
an inner filler material;
wherein said inner filler material is formed from a fiber material;
wherein said inner filler material is surrounded by said outer layer and enclosed therein;
wherein said improved ear plug is formed having a predetermined size, shape, and stiffness; and
wherein said improved ear plug is adapted to be removably placed adjacent to or within the ear canal of a user's ear and adapted to prevent and relieve symptoms caused by changes in barometric air pressure, cold temperatures, or windy conditions.

2. The improved ear plug of claim 1, wherein said gauze material is a cotton gauze material.

3. The improved ear plug of claim 1, wherein said fiber material is a tissue fiber material.

4. The improved ear plug of claim 1, wherein said tissue fiber material is formed from toilet paper.

5. The improved ear plug of claim 1, wherein said gauze material and said inner filler material are sized and shaped such that said inner filler material can be wrapped within said gauze material, and then edges of said gauze material connected together thereby resulting in said predetermined size, shape, and stiffness.
